(19) United States
(12) Patent Application Publication
(10) Pub. No.: US 2020/0159954 A1
(43) Pub. Date: May 21, 2020

(54) Method Of Processing Document Requests

(71) Applicants: Andrew Pomerance, Alexandria, VA (US); Colin McCann, Arlington, VA (US); William Pomerance, Alexandria, VA (US)

(72) Inventors: Andrew Pomerance, Alexandria, VA (US); Colin McCann, Arlington, VA (US); William Pomerance, Alexandria, VA (US)

(21) Appl. No.: 16/430,052

(22) Filed: Jun. 3, 2019

Related U.S. Application Data
(60) Provisional application No. 62/679,530, filed on Jun. 1, 2018.

(51) Int. Cl. G06F 21/62; G16H 10/60; G16H 40/20

(52) U.S. Cl. CPC G06F 21/6245; G16H 40/20; G16H 10/60

(57) ABSTRACT

A method for processing record requests on an app running on a computing device includes entering user information, identify the records, validating the user, determining if the app is running on a device having a specific functionality, selectively instructing the user to provide verifying information based on the determined device functionality, validating the verifying information and providing the records to the user.

### BACKGROUND

The present disclosure is directed to electronic records and more particularly to processing requests for electronic medical records.

Electronic records are well known. As society evolved from manual record keeping with the advent of computers and inexpensive storage, more and more records have been maintained in electronic form. With advances in networking and cloud storage, virtually all records are now in electronic form.

One type of records that is highly shielded is a medical record. Confidentiality of medical records is a high priority. Consequentially, obtaining one's own medical records is rather cumbersome and inefficient. Currently, a user is often required to either show up in person at a health care facility (e.g. hospital, lab or doctor's office) to request copies of his or her records or submitting such request by facsimile.

As virtually everyone appears to be equipped with mobile devices (i.e. cell phones), an increasing percent of transactions are taking place via this device.

It is desirable, therefore, to provide a system and method for facilitating requests for obtaining electronic records in a secure and efficient manner.

### SUMMARY

According to an exemplary embodiment, a method for processing record requests on an app running on a computing device is disclosed. The method comprises: entering user information; specifying the records; validating the user; determining if the app is running on a device having a specific functionality; selectively instructing the user to provide verifying information based on the determined device functionality; validating the verifying information; and providing the records to the user.

According to another exemplary embodiment, a system for processing requests for records is disclosed. The system comprises: a computing device having an app running thereon; a server; and a network connecting the device to the server. The server comprises a memory having records stored therein; and the app provides a user interface between a user and the server. The user interface receives user information for validation by the server. The received information includes user identity and identity of the requested records. The user interface provides the requested records to the user.

### BRIEF DESCRIPTION OF THE DRAWINGS

The several features, objects, and advantages of exemplary embodiments will be understood by reading this description in conjunction with the drawings. The same reference numbers in different drawings identify the same or similar elements. In the drawings:

FIG. 1 illustrates a method in accordance with an exemplary embodiment; and

FIG. 2 illustrates a method in accordance with another exemplary embodiment;

FIG. 3 illustrates a system in accordance with exemplary embodiments; and

FIGS. 4-7 illustrate exemplary user interfaces.

### DETAILED DESCRIPTION

In the following description, numerous specific details are given to provide a thorough understanding of exemplary embodiments. The embodiments can be practiced without one or more of the specific details, or with other methods, components, etc. In other instances, well-known structures, or operations are not shown or described in detail to avoid obscuring aspects of the exemplary embodiments.

Reference throughout this specification to an “exemplary embodiment” or “exemplary embodiments” means that a particular feature, structure, or characteristic as described is included in at least one embodiment. Thus, the appearances of these terms and similar phrases in various places throughout this specification are not necessarily all referring to the same embodiment. Furthermore, the particular features, structures, or characteristics may be combined in any suitable manner in one or more embodiments. The headings provided herein are for convenience only and do not interpret the scope or meaning of the embodiments.

A method for processing requests for records is described in accordance with exemplary embodiments. The records may be electronic medical records stored in a database on a server accessible over a network for example. The database or server may be associated with a particular hospital or hospital chain or a medical clinic. The server may physically be collocated with the associated facility or be in the cloud.

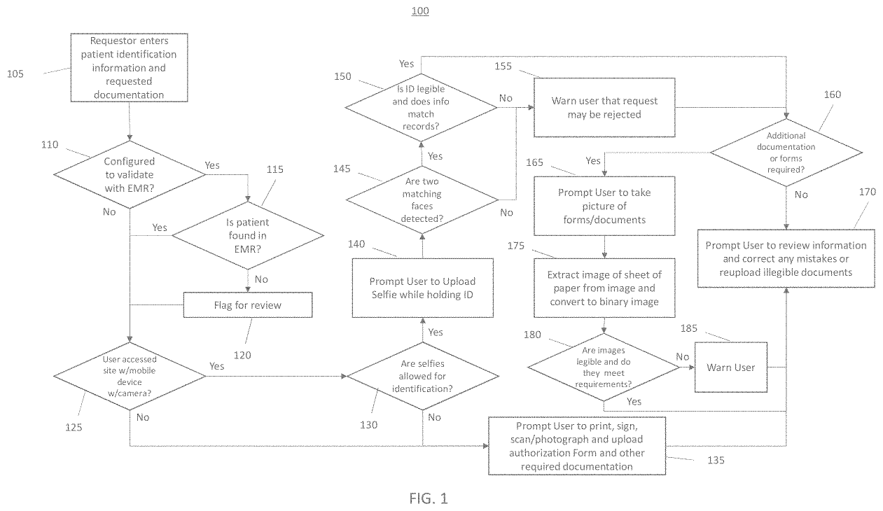

An exemplary method is described in conjunction with the process illustrated in FIG. 1. A requestor (such as a patient) enters his or her identification information at 105. In order to process the request, a determination is made as to whether the server receiving the request is configured to directly validate the request in the facility's Electronic Medical Record (EMR) at 110. This determination is based on the security policies of the healthcare facility.

If the server is not configured to validate with EMR (Electronic Medical Records), the process continues to 125 at which a determination is made as to whether the user (patient) accessed the server using a mobile communication device (i.e. a cell phone) having image capturing capability (i.e. a camera).

If the server is configured to validate with EMR (Electronic Medical Records), a determination is made as to whether the patient records are exist in EMR at 115. If the patient records are found, the process continues to 125 at which a determination is made as to whether the user (patient) accessed the server using a mobile communication device (i.e. a cell phone) having image capturing capability (i.e. a camera).

If the patient records are not found (at 115), the request is flagged for review at 120 and the process continues to 125 at which a determination is made as to whether the user (patient) accessed the server using a mobile communication device (i.e. a cell phone) having image capturing capability (i.e. a camera).

If the user access is via a cell phone or other device with a camera (at 125), a determination is made as to whether selfies are allowed (or accepted by the server) for identification at 130. This determination depends on the security policies of the specific healthcare facility. If selfies are not allowed, the user may be prompted to print, sign, scan and upload an authorization form and any other pre-determined required information at 135. The user may also be prompted to print, sign, scan and upload an authorization form and any other pre-determined required information at 135 if it is determined that the user did not access via a cell phone with a camera (at 125). The process continues to 170 at which the user may be prompted to review information and correct any mistakes or “re-upload” any illegible documents as determined by the user.

If selfies are allowed (at 130), the user may be prompted to capture a selfie while holding an identification of the user and uploading or submitting the selfie at 140. The submission may be via a text message or as an e-mail attachment or by a mobile internet connection with the server. A determination may be made at 145 (by the server) as to whether two matching faces are detected in the submitted selfie. One face may correspond to the user as captured by the cell phone camera and the other face may correspond to the face on the user identification also captured by the cell phone camera. If two faces are not detected, the user may be warned that the request for records may be rejected at 155.

If two faces are detected, a determination may be made at 150 as to whether the identification is legible (and whether the information matches records). If identification is legible (and information matches records), the process continues to 160 at which a determination may be made as to whether additional documents or forms are required.

If identification is not legible (and/or information does not match records), the user may be warned that the request for records may be rejected at 155. At 160, a determination may be made as to whether additional documents or forms are required.

If it is determined that no additional documentation or forms are required, the user may be prompted to review information and correct any mistakes or “re-upload” any illegible documents as determined by the user at 170.

If the determination is made (at 160) that additional documentation or forms are required, the user may be prompted to take a picture of the documents or forms to submit to the server at 165. The image or picture may be submitted via a text message or as an e-mail attachment or by direct connection via mobile internet. An image of the picture may be converted to a binary image at 175 by a processor associated with the server. The extracted image(s) may be evaluated to determine if the extracted images are legible and whether they meet pre-determined requirements for acceptable document files at 180.

If they are illegible or if they do not meet the requirements, the user may be warned or informed of the inadequacy of the extracted image at 185. The user may then be prompted to review information and correct any mistakes or “re-upload” any illegible documents at 170 as determined by the user.

If they are legible and deemed to meet the requirements (at 180), the user may be similarly prompted at 170 to review information and correct any mistakes or “re-upload” any illegible documents as determined by the user.

If the user is verified/validated as described above, the requested documents may be provided to the user instantly in an electronic format. The requested documents may also be submitted to the user via mail or by facsimile.

In order to comply with existing legal requirements, each request as validated above may also be reviewed and approved by a reviewing entity prior to retrieving and submitting the documents to the requesting user. The reviewer may be an individual. In some embodiments, artificial intelligence systems can be utilized to perform the approval process. Such systems can incorporate machine learning to further refine the approval process.

The user may be responsible for payment of fees associated with obtaining the records. Such payments can be processed utilizing known methods.

In another exemplary method, a request for filling out forms is disclosed. This may occur if a user is claiming disability for example. The user's employer or insurance company may require proof of disability by providing supporting health records, etc. In such cases, the user may be required to have his or her doctor or health care provider fill out forms to support the user's claim(s).

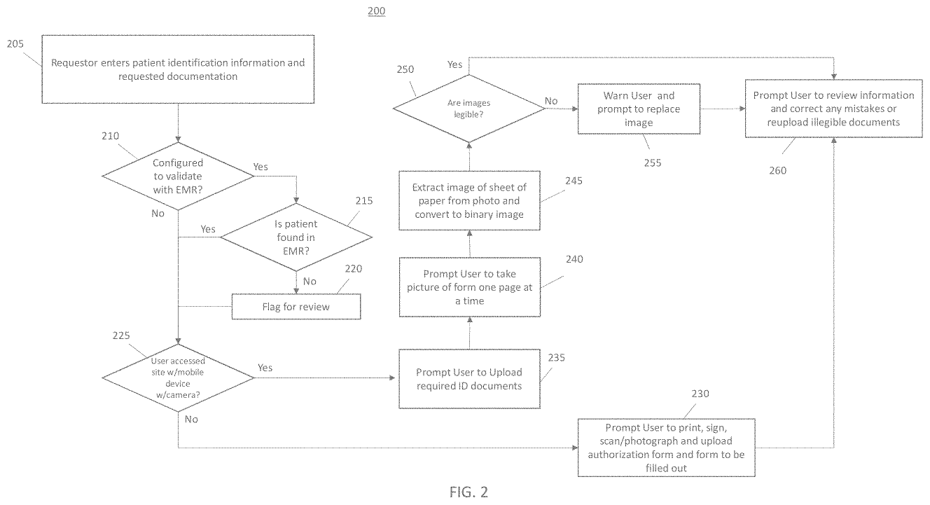

A method in accordance with exemplary embodiments for filling out forms is described in conjunction with the process illustrated in FIG. 2. At 205, a requestor (such as a patient) enters his or her identification information. In order to process the request, a determination is made as to whether the system receiving the request is configured to validate the request at 210.

If the system is not configured to validate with EMR (Electronic Medical Records), a determination may be made as to whether the user (patient) accessed the server using a mobile communication device (i.e. a cell phone) having image capturing capability (i.e. a camera) at 225.

If the system is configured to validate with EMR (Electronic Medical Records), a determination is made as to whether the patient records exist in EMR at 215. If the patient is not found, the request is flagged for review at 220. The process continues to 225 where a determination may be made as to whether the user accessed the server using a cell phone having a camera.

If the patient is found in EMR, the process continues to 225 where a determination may be made as to whether the user accessed the server using a cell phone having a camera.

If the user did not access the records database with a cell phone having a camera, the user may be prompted to print, sign, scan and upload an authorization form to be filled at 230. The user may be prompted to review information and correct any mistakes or “re-upload” the documents that may be illegible at 260.

If the user did access the records database using a cell phone having a camera (at 225), the user may be prompted to upload required identification documents at 235. The user may be prompted to capture an image (i.e. take a picture using the mobile phone) of the form one page at a time at 240. The images of the forms may be uploaded as a text message or as e-mail attachments, etc. The image of a sheet of the forms may be extracted from the photo/picture and converted to a binary image at 245 by a processor associated with the server.

A determination is made as to whether the images are legible at 250. If the images are determined to be not legible, the user may be warned and prompted to replace the images at 255. The user may be prompted to review information and correct any mistakes or “re-upload” the documents that may be illegible at 260.

If the images are determined to be legible (at 250), the user may be prompted to review information and correct any mistakes or “re-upload” the documents that may be illegible at 260.

The requests as validated may be reviewed as described above. In this embodiment, the received forms may be filled out as needed. Any fees that may be associated with providing the records or forms may be processed as described above.

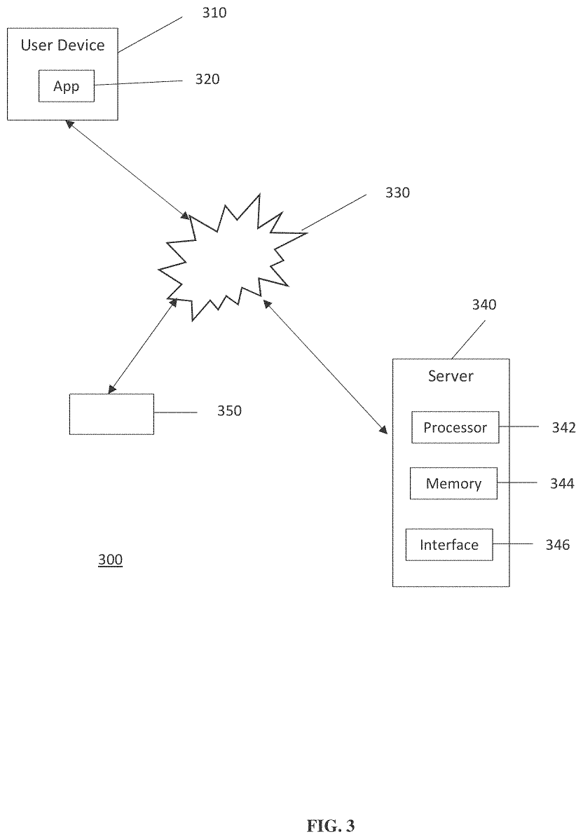

A system 300 in accordance with exemplary embodiments is illustrated in FIG. 3. System 300 may include a communication computing device 310 such as a mobile phone or a tablet. Device 310 may include a mobile application (“app”) 320 which is a computer program or software application. Various modules, components and features (hardware, software, user interface, display, memory, camera, processor, antenna, modem, etc.) of a mobile phone are known and are not described further.

System 300 may also include a server 340. Server 340 may be connected to device 310 via network 330. Network 330 may be a private network or a public network. Device 310 may connect with network 330 via a physical (wired) link or a wireless link such as a wi-fi or a mobile communication interface or the like.

Server 340 may include a processor 342, a memory 344, a communication interface 346 and other known components, modules and interfaces. Memory 344 may have stored therein electronic records such as the medical records.

Server 340 may also communicate with a computing/communication device 350 over network 330. Device 350 may be associated with a verification and/or validation entity. Device may include a display, a memory, a user (input) interface, a communication module and other known components associated with computer for example.

A user may enter information using computing device 310. Information may be entered using the input interface and the camera may be used to take a selfie with identifying information as described above.

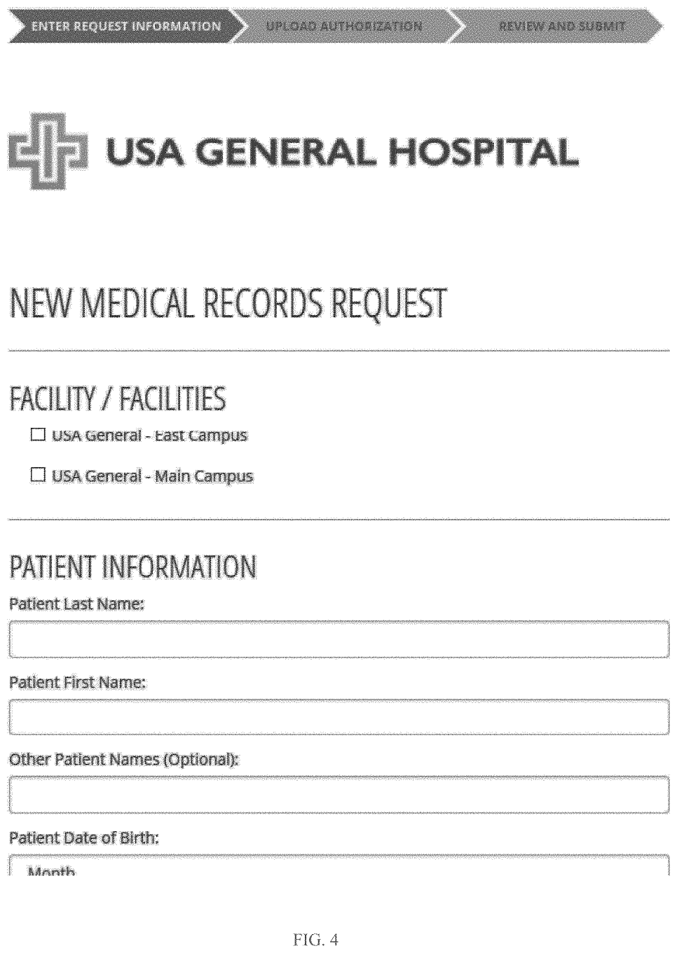

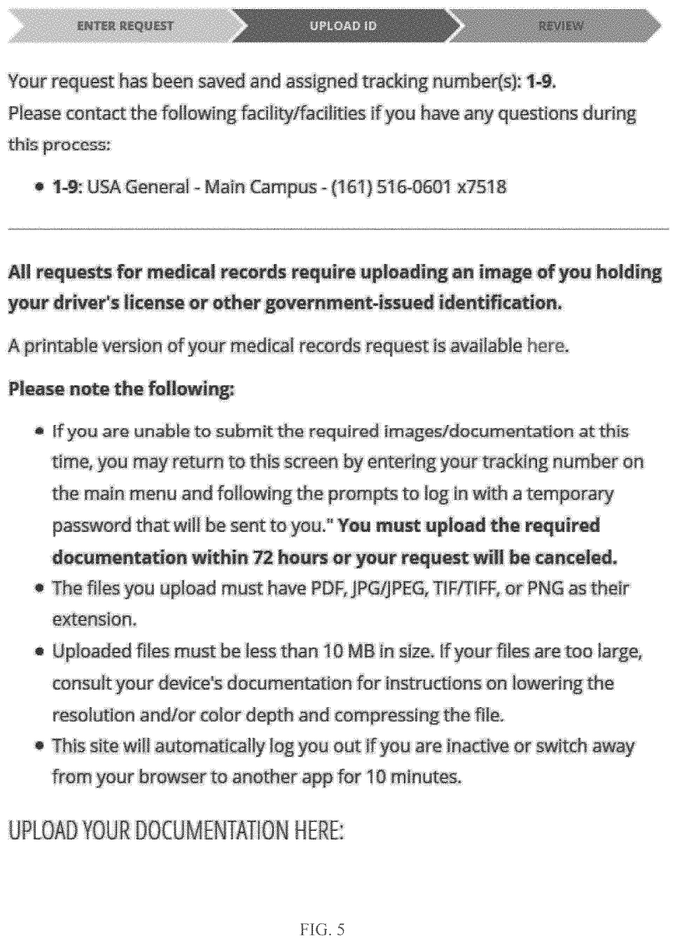

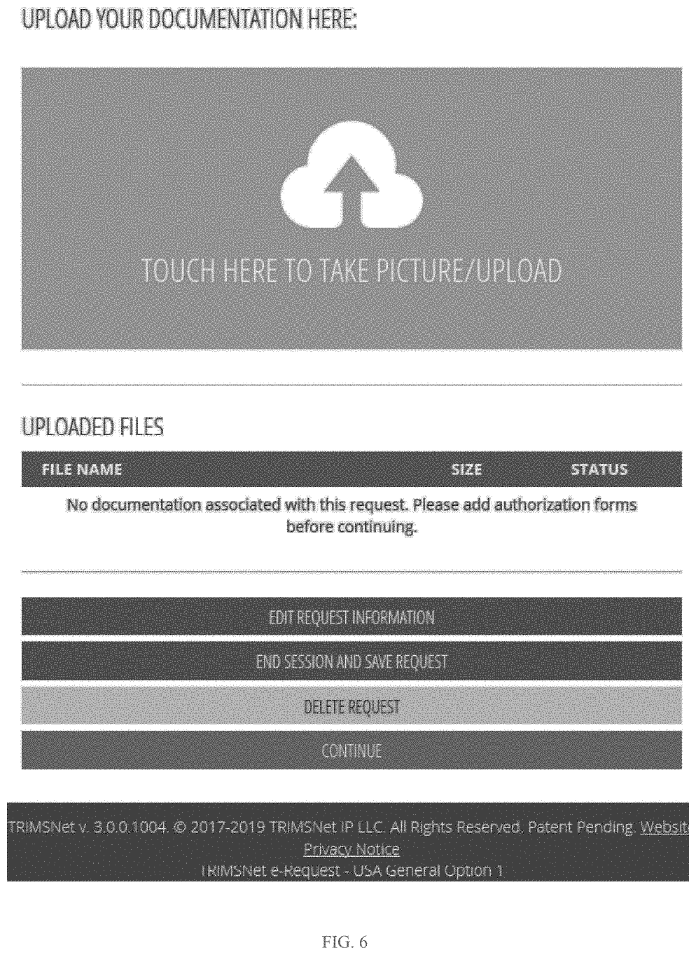

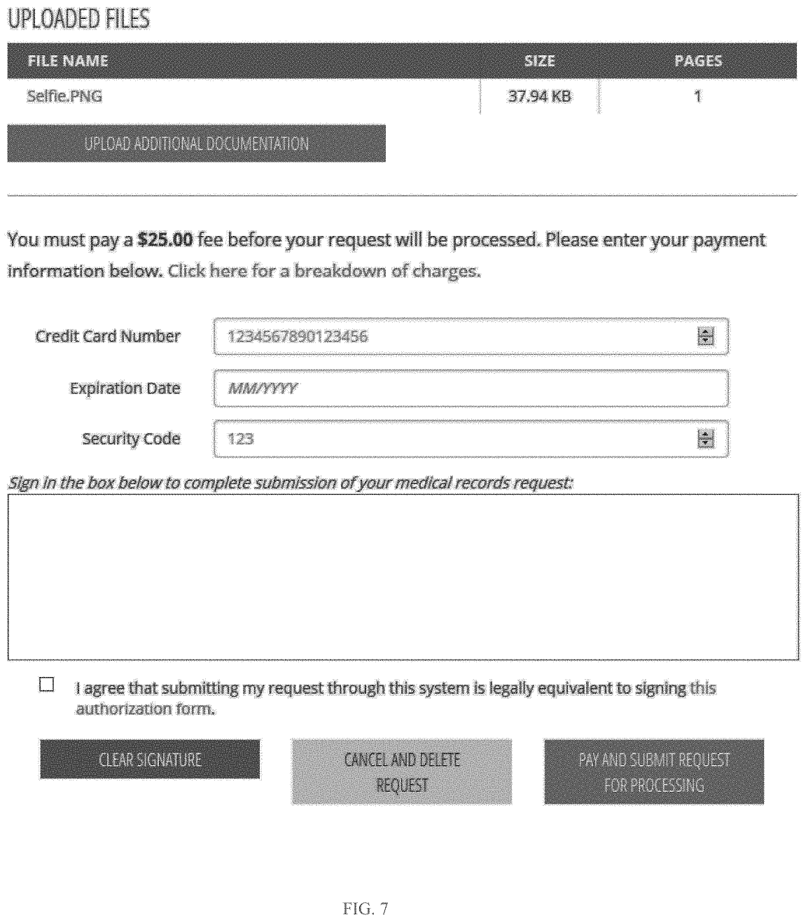

An application (“app”) 320 may be executed on device 310. A user interface associated with the app for facilitating user entry may appear as illustrated in FIG. 4. Information about the process/procedure for obtaining the records may be displayed as illustrated in FIG. 5. Instructions for taking a selfie or providing options to a user such as editing request information, ending session and saving request, deleting request or continuing may be displayed as illustrated in FIG. 6. Instructions to pay for the documents, signing the request, clearing signature, cancelling and deleting request and paying/submitting request may be displayed as illustrated in FIG. 7.

Exemplary embodiments describe a method and a system for validating, verifying and securely processing requests for records. Records may be in an electronic form. They may be medical records. Methods as described above can also be implemented on a laptop or on a desktop computer.

While the foregoing disclosure enables one of ordinary skill to make and use what is considered presently to be the best mode thereof, those of ordinary skill will understand and appreciate the existence of variations, combinations, and equivalents of the specific embodiment, method, and examples herein. The description should therefore not be limited by the above described embodiments, methods and examples, but by all embodiments and methods within the scope and spirit of the disclosure. All such embodiments are intended to be covered by the appended claims.

Further, in the description and the appended claims the meaning of “comprising” is not to be understood as excluding other elements or steps. Further, “a” or “an” does not exclude a plurality, and a single unit may fulfill the functions of several means recited in the claims.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for processing document requests on an app running on a computing device, the method comprising:
entering user information;
specifying information sought;
validating the user;
determining if the app is running on a device having a specific functionality;
selectively instructing the user to provide verifying information based on the determined device functionality;
validating the verifying information; and
providing the records to the user.

11. A system for processing document requests, the system comprising:
a computing device having an app running thereon;
a server; and
a network facilitating connection of the device to the server wherein
the server comprises a memory having records stored therein; and
the computing device comprises an app providing a user interface between a user and the server wherein the user interface
receives user information for validation by the server, said information including user identity and specific documents; and
provides the specified documents to the user.
